(19) United States
(12) Patent Application Publication
(10) Pub. No.: US 2010/0121250 A1
(43) Pub. Date: May 13, 2010

(54) Dual Channel Shunt Device and Method for Ventriculo-Peritoneal Shunting of Bloody Cerebrospinal Fluid

(76) Inventor: Francis J. Pizzi, Princeton, NJ (US)

Correspondence Address: FOX ROTHSCHILD LLP, 997 Lenox Drive, Building 3, Lawrenceville, NJ, 08648, US

(21) Appl. No.: 11/993,617

(22) PCT Filed: Feb. 7, 2007
(86) PCT No.: PCT/US07/61747

§ 371 Date: Dec. 21, 2007

Related U.S. Application Data
(60) Provisional application No. 60/771,691, filed on Feb. 9, 2006.
(60) Provisional application No. 60/804,659, filed on Jun. 14, 2006.

(51) Int. Cl. A61M 1/00

(52) U.S. Cl. 604/10; 604/9

(57) ABSTRACT

A dual channel shunt device (30) and method allow for the easy and safe draining of first bloody and then clear hydrocephalic fluid (28) from the brain. A burr hole (22) is drilled to allow a catheter (14) to access the lateral ventricle (12) and then a tube (26) is run under the scalp (26) to the dual channel device (30) and then from the dual channel device (30) through another tube (18) to drain into the peritoneal cavity (36). The first channel (26a, 26b) comprises an on-off switch (32,60) coupled with an anti-siphon device (64). The second channel (26b, 18b) comprises a pressure resistant valve (34) offering a resistance in the range of 40-80 Millimeters of water. Initially the bloody fluid (28) is drained through the on/off switch (32). When the fluid (28) has cleared, as determined by a CT scan, the surgeon turns off the on-off switch (32) thereby diverting the clear fluid (28) through the pressure resistant valve (34) into the peritoneal cavity (36).

### CROSS-REFERENCE TO RELATED INVENTIONS

This application is based on and claims the priority of U.S. Provisional Patent Application No. 60/771,691 filed on Feb. 9, 2006 and entitled “Device of Ventriculo-Peritoneal Shunting of Bloody Cerebrospinal Fluid” and U.S. Provisional Patent Application No. 60/804,659 filed on Jun. 14, 2006 and also entitled “Device of Ventriculo-Peritoneal Shunting of Bloody Cerebrospinal Fluid” both by inventor Francis J. Pizzi, the entire contents and substance of both of which are hereby incorporated in total by reference.

### BACKGROUND OF THE INVENTION

### Description of Related Art

The incidence of obstructive hydrocephalus caused by a spontaneous hemorrhage in the brain is currently in excess of 60,000 cases per year in the USA. This number is increasing due to the more prevalent usage of blood thinners (coumadin, heparin, herbal remedies, Vitamin E Supplement) and platelet inhibitors (aspirin, Plavix®) for cardiac conditions and stroke prevention in the aging baby boomer population. A patient on these medications has a far greater chance of developing a hemorrhagic stroke versus a non-hemorrhagic stroke from a brain vessel occlusion. Additionally, minor head trauma in these patients is more likely to cause a bruise to the brain with bleeding into the cerebrospinal fluid pathways.

On an almost daily basis, the average neurosurgeon is confronted with an acutely ill patient who has sustained a spontaneous hemorrhage in their brain. Most of the time, these hemorrhages will extend into the cerebrospinal fluid system. In this system, fluid is produced at a rate of 20 cc. per hour in the lateral ventricles and must make its way through a series of passages of very small diameter to outflow at the junction of the brain and spinal cord. This fluid then is resorbed at a rate of 20 cc. per hour by the venous system which transports it to the kidneys where it is excreted. Blood particles in this pathway may clog up the smaller passages preventing the fluid from reaching the area where it is resorbed. The fluid continues to be produced but cannot be resorbed, thus an accumulation of the bloody fluid distends the ventricles, pouches deep in the hemispheres of the brain. The distended ventricles put pressure on the brain and the patient slips into a coma and dies.

The neurosurgeon must do something to relieve the pressure in the brain caused by the obstructive hydrocephalus. One option is to perform a ventriculo-peritoneal shunt using a tube with an integrated one way valve as shown in FIG. 1A. One end of the tube is inserted through a hole in the skull and is passed through the substance of the brain into the cavity or ventricle of the brain where the fluid is produced. This tube is then tunneled under the scalp to the one way valve and then through another subcutaneous tunnel to the peritoneal cavity of the abdomen. Here the fluid drains and is absorbed by the lining, then absorbed by the veins of the lining and transported to the kidneys for excretion. Neurosurgeons have been doing this procedure successfully for over 50 years utilizing many different valved systems to treat hydrocephalus that is not associated with blood in the cerebrospinal fluid. In the presence of blood within the spinal fluid, these valved shunt systems commonly fail due to blood particles blocking the very small passages in the valve. With shunt blockage by blood, the hydrocephalus will once again recur to jeopardize the brain and re-operation is mandated.

The life saving treatment of choice by the neurosurgeon for the vast majority of patients with obstructive hydrocephalus secondary to blood particle obstruction of the cerebro-spinal fluid pathways is External Ventricular Drainage (EVD) as shown in FIG. 1B. This involves making a hole in the skull, inserting a tube through the brain substance into the ventricle, and attaching it to an external bag where the bloody fluid drains. A staff member periodically empties the fluid as shown in FIG. 1C. The amount of fluid drained is regulated by positioning the tube coming from the ventricle at a level about 10 cm. higher than the patients head as shown in FIG. 1B. This creates a hydrostatic pressure so that any time the fluid pressure inside the brain exceeds 10 cm. of water pressure (normal), fluid will drain. Fluid continues to be produced in the ventricles at 20 cc. per hour. This will eventually dilute the bloody fluid as can be seen by observing the transparent drainage tube. At this time the neurosurgeon has the choice of removing the EVD (with the hope that the blood particles obstructing the fluid passages in the brain have cleared) or performing the above described ventriculo-peritoneal shunt as shown in FIG. 1A.

There are prior art patents and literature references to devices which relate, in some respects, to the present invention. For example, U.S. Pat. No. 4,781,673 describes a “Brain ventricle shunt system with flow-rate switching mechanism”. This patent is related in that dual channels leading to valves of different flow rate can be isolated with an on-off switch. The device allows the neurosurgeon to choose one of two valves of different flow rates with tubes leading either to the atrium of the heart or to the peritoneal cavity. In that valves are present which can become blocked with blood particles, this would not be applicable to a patient with obstructive hydrocephalus secondary to bloody cerebrospinal fluid.

Likewise, U.S. Pat. No. 5,154,693 describes a “Flow control device having selectable alternative fluid pathways”. This allows choice of one of two valved pathways to select appropriate flow rates to drain fluid in the hydrocephalic patient in the absence of blood in the cerebrospinal fluid. See also U.S. Pat. No. 5,167,615.

Certain isolated features of the present invention and method are also known in the prior art. For example, an on-off switch as shown in FIG. 6B is described in the patent literature as early as in U.S. Pat. No. 3,827,439. Anti-siphon devices are also known in the literature and described, for example, in U.S. Pat. No. 4,795,437 and 6,953,443.

A more recent iteration of the above is U.S. Pat. No. 5,167,615 which adds a magnetically controlled switch to allow selection of pathway to a flow valve. None of these devices or methods solves the problem of obstructive hydrocephalus secondary to blood in the cerebrospinal fluid pathways.

There are many deficiencies and patient risks associated with the device and method (see FIG. 1C) of external ventricular drainage (EVD) which is the current art for treating obstructive hydrocephalus due to hemorrhage into the cerebro-spinal fluid pathways of the brain.

Life threatening meningitis (infection of the outer coverings of the brain) and or ventriculitis (infection of the inner lining of the ventricle) occurs in excess of 11% of patients treated with the current art in one series. See Wilberger, J. E., et al. Neurosurgery, 27:208-213, 1990. In a more recent study there was a 7% infection rate. See Mayhall, C. G., et al. N. Engl. J. Med., 310:553-559, 1984. All studies agree that the longer an EVD is left in the ventricle, the higher the infection rate. Infection is caused by establishment of a fluid pathway between the cavity of the brain and the hospital ICU (Intensive Care Unit) environment which is contaminated with many diverse bacterial pathogens.

An EVD is external to the body and can become dislodged. Commonly a patient with an impaired level of consciousness is restless and sometimes combative. These patient motions can cause dislodgement of the tube in the brain cavity and it will no longer function properly. Additionally, hospital staff, when moving a poorly responsive patient in their bed, can accidentally dislodge the drain tube. In both of these circumstances, re-operation is necessary to replace the drain catheter to relieve pressure on the brain. Re-operation requires passing a tube through the substance of the brain into the ventricle. Each time this is done there is a 1.4% chance of causing a hemorrhage into the substance of the brain. See Narayan, R. K., et al. J. Neurosurg., 56:650-659, 1982

Staff mishandling of the EVD can cause over drainage of fluid from the ventricle due to siphoning. This will cause the brain to collapse away from the inner table of the skull. The bridging veins from the brain surface to the main draining veins that are attached to the skull are torn away and begin hemorrhaging over the brain surface. This causes a subdural hematoma which must be surgically drained.

When the fluid issuing through the EVD begins to clear from the dilution resulting from continuous production of cerebrospinal fluid in the ventricle, the neurosurgeon may choose to remove the tube. If hydrocephalus recurs from premature removal or a new bleed, the EVD must be replaced in another operation with a 1.4% chance of the procedure causing a hemorrhage into the substance of the brain. Blood when outside the blood vessels acts as an irritant to the brain and irritation causes scar tissue. After the EVD is removed, delayed hydrocephalus can occur due to scar tissue formed in the CSF pathways. A ventriculo-peritoneal shunt operation must be performed, usually on an emergent basis, to save the patient's life.

### SUMMARY OF THE INVENTION

The proposed method and device, Dual Channel IVD (Internal Ventricular Drain)/Shunt, effectively eliminates deficiencies and patient jeopardies associated with the usage of the External Ventricular Drainage (EVD) method and device in the presence of obstructive hydrocephalus secondary to blood in the cerebrospinal fluid pathways.

A tube inserted in the ventricle through a hole in the skull is run under the scalp to an implanted device that receives the bloody ventricular fluid. The device splits into two channels, one with an on-off switch and the other with a ball-in-cone spring valve. The “on” position allows the bloody fluid to run in a subcutaneously implanted catheter directly to the peritoneal body cavity for re-absorption passing through an anti-siphon device with no interposed valves that could become blocked with blood particles. When the CT scans show the fluid becoming less bloody through normal dilution, the scalp over the switch is palpated into the “off” position thus diverting the diluted fluid to a one way pressure valve which then connects to the catheter going to the peritoneal cavity.

### BRIEF DESCRIPTION OF THE DRAWINGS

FIG. 1A is a diagram of a prior art ventriculo-peritoneal shunt.

FIG. 1B is a diagram of a prior art External Ventricular Drain (EVD).

FIG. 1C is a flow diagram illustrating the steps used to perform the prior art technique shown in FIG. 1B.

FIG. 2 is a diagram of the preferred embodiment of the Dual Channel Shunt of the present invention for draining cerebrospinal fluid implanted in a patient.

FIG. 3 is a flow diagram illustrating the steps used to perform the preferred embodiment of the present invention as shown in FIG. 2

FIG. 4 illustrates the preferred embodiment of the Dual Channel shunt shown in FIG. 2.

FIG. 5A illustrates the initial path of the bloody fluid through the on-off switch of the dual channel shunt.

FIG. 5B illustrates the path of the fluid after it has been determined by CT scan to be diluted through the pressure resistant ball-in-cone valve and after the on-off switch of the dual channel shunt has been switched off.

FIG. 6A is detailed crossed sectional view of a pressure resistant ball-in-cone valve.

FIG. 6B is a detailed cross-sectional view of an on-off switch including a reservoir and an anti-siphon device.

FIGS. 7A-7E illustrate the typical steps taken to turn the on-off switch off or on and how to flush it.

### DESCRIPTION OF THE PREFERRED EMBODIMENT OF THE INVENTION

During the course of this description like numbers will be used to identify like elements according to different figures that illustrate the invention.

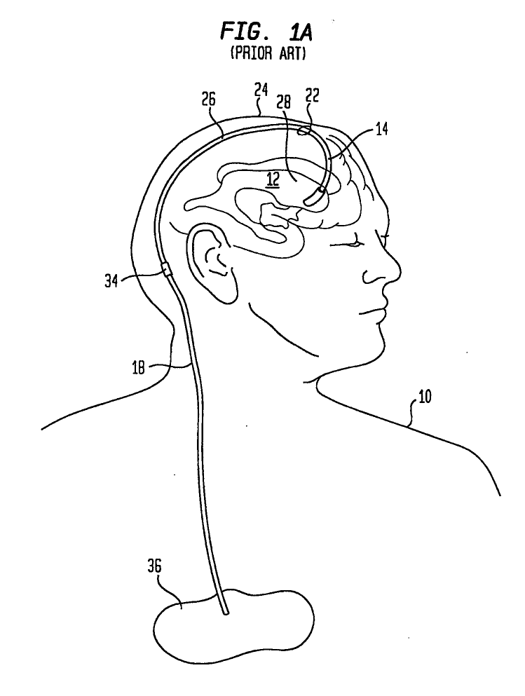

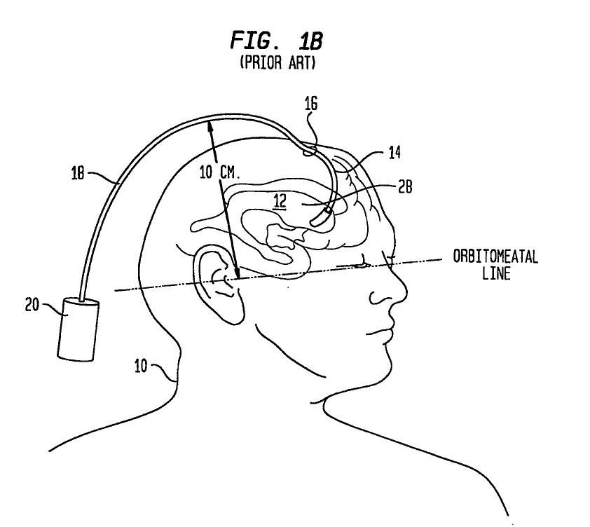

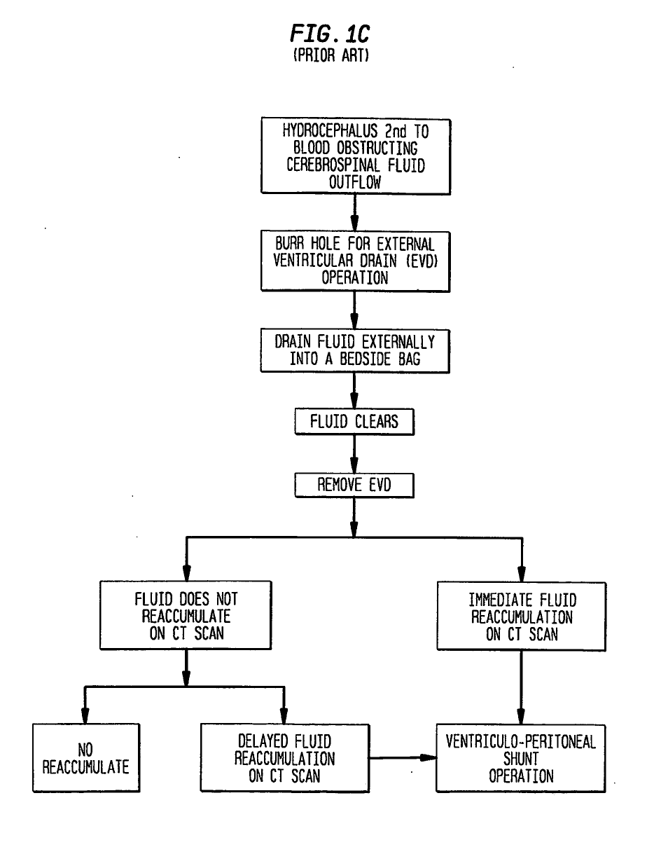

Fluid (28) build up in the cavities of the brain, known as hydrocephalus, is familiar to lay people but is usually thought of as an affliction of a newborn child. Hydrocephalus also occurs in adults for various reasons. Neurosurgeons have been treating hydrocephalus in various ways for more than 50 years. The operative procedure of choice for both children and adults is called “Ventriculo-Peritoneal Shunt”. This prior art technique is illustrated in FIG. 1A. Initially a burr hole 22 is drilled through the skull of the patient 10. A ventricular catheter 14 is then inserted through the skull hole then through the brain substance into the lateral ventricle 12. That is in turn connected to a valve that opens at a pre-set pressure to allow fluid 28 to drain through tube 26 when the pressure in the lateral ventricle 12 is greater than the pressure in the valve 34. This tube is the passed through a tunnel under the skin to another incision in the upper abdominal area where the tube 18 is implanted in the peritoneal cavity 36. The lining of the abdominal cavity 36, the peritoneum, will absorb the fluid 28 and return it to circulation through the veins. The fluid 28 from the ventricle 12 now has another pathway to drain out without building up pressure and damaging the brain. The valve prevents too much fluid from draining and the consequences thereof. The steps of the prior art approach are graphically illustrated in FIG. 1A.

The prior art system will work well provided that the fluid in the brain cavities is normal in protein content and does not contain blood. If blood or fluid with high protein content is present, the plastic tubing and/or the valve will become clogged. The shunt will fail causing a build up of fluid in the brain cavity and hydrocephalus with subsequent death.

Commonly, the patients will present emergently with the new onset of hydrocephalus due to a hemorrhage into the brain cavity from a stroke or from a brain injury. To save a person's life in these circumstances, the neurosurgeon will drill a hole 22 in the skull, insert a plastic tube 14 into the brain cavity 12 and allow the cerebrospinal fluid to drain externally into a bag 20. This is called External Ventricular Drainage or EVD as shown in FIG. 1B.

EVD solves the immediate life threatening situation but not the long term problem. The drain tube can only be left in place for a limited time because there is the constant threat of hospital acquired infection causing meningitis and/or ventriculitis, an infection of the lining of the ventricle cavity deep in the brain. The plastic tube and the fluid column within it establish a pathway for germs from the hospital environment to get into the brain through the drill hole in the skull. Additionally, a patent may become restless and combative as a result of their condition. It is not uncommon for a patient to accidentally pull the external drain out of their ventricle thus necessitating a replacement operation.

The neurosurgeon observes the fluid that drains from the brain cavity for several days. See FIG. 1C. When the fluid has visually cleared of blood, the drain is either removed or a ventriculo-peritoneal shunt operation is performed to correct the long term problem. There is a greater likelihood that this newly implanted shunt, when done after several days of open external drainage, will become infected. This would then require removal of the shunt, external drainage again and the implantation of yet another shunt after the infection has cleared, an additional three surgeries. This chain of events can repeat itself again and again.

The present invention 30 is specifically intended to treat patients with hydrocephalus associated with bloody cerebrospinal fluid. External ventricular drainage is unnecessary and subsequent ventriculo-peritoneal shunting as a second procedure is unnecessary. Exposure to the risks of ventriculitis, meningitis, hemorrhage into the brain substance and multiple surgeries under anesthesia is eliminated.

To better understand the present invention 30 it is first helpful to understand the anatomy, physiology and pathology of the environment. There are several separate circulation systems in the human body. The most familiar of these is the blood system with its arteries and veins. The cerebrospinal fluid circulation system involves the production of a clear watery fluid in the brain cavities. This fluid is produced at a constant rate of 0.34 milliliters per minute or about 20 mm (four teaspoonfuls) per hour. This fluid must get out of the brain to be reabsorbed in the spinal canal; hence it is called cerebrospinal fluid. There are pouches deep within the brain substance which drain through openings that join in the mid-line at the third ventricle which is a narrow slit. The fluid then drains through a one millimeter diameter passageway through the center of the brain to emerge in the fourth ventricle. This drains to the upper spinal canal through tiny openings and then flows down the spinal canal where it is absorbed to go back into the veins.

If any of the passages, openings or tubes through which the fluid must traverse to get out of the brain become blocked or clogged, the fluid cannot emerge. Blood in the cerebrospinal fluid is notorious for doing this. The production of fluid continues at its same rate despite the blockage. Thus the cavities of the brain become enlarged and they put pressure on the surrounding brain tissue. The brain can accommodate this increased pressure for a time but eventually cannot, at which time the brain stops functioning. The patient lapses into a coma and shortly will die unless the pressure is relieved.

The most common cause of acute hydrocephalus is bloody cerebrospinal fluid. The most common causes of bloody cerebrospinal fluid are head injuries and hemorrhagic strokes. The blood clogs up the passageways through which the fluid produced in the brain must egress to get to the site of absorption in the spinal canal.

The incidence of hemorrhagic strokes is definitely increasing. There are several reasons for this. Life spans are increasing for men and women. The baby boomer population glut is entering the years when strokes occur. The greatest factor, however, is the aggressive treatment of people with anti-coagulants (heparin, coumadin, etc.) and platelet inhibitors (Plavix®, etc.) to treat cardiac conditions, partly blocked arteries and mini-strokes. Those who take medicines to reduce their chances of having a stroke can have a stroke despite this precaution. Compared to those who do not take anti-coagulants and/or platelet inhibitors they are much more likely to hemorrhage into the damaged area of the brain where the stroke occurred. This hemorrhage into the damaged area frequently extends into the ventricles causing bloody cerebrospinal fluid.

When people taking these medications sustain even a mild head injury they are prone to have a brain hemorrhage. When the head is struck or even shaken, the brain, which floats in the cerebrospinal fluid inside the skull sustains bruises from hitting the hard bone that protects it. We all know how even mild blood thinners such as aspirin can cause a minimal skin bruise to blossom into a large purple blotch. The same thing happens to the much more fragile brain surface. Blood from the bruise gets into the cerebrospinal fluid pathways and can cause a blockage of fluid flow with resultant hydrocephalus.

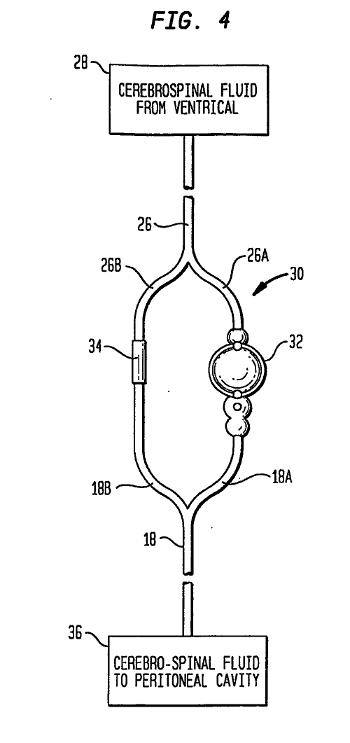

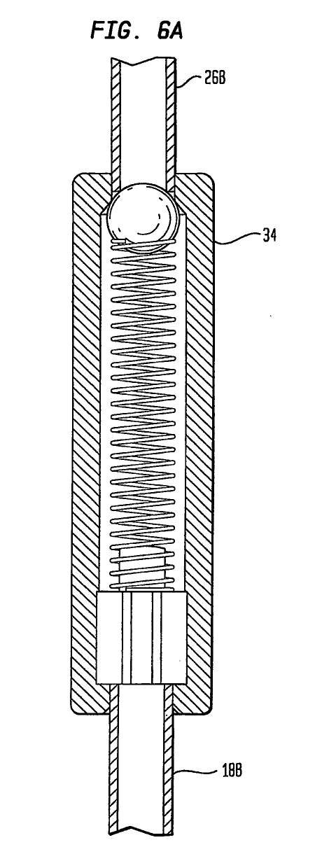

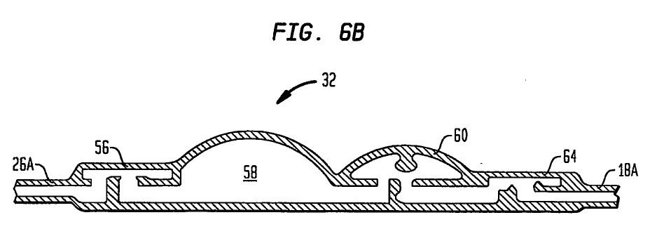

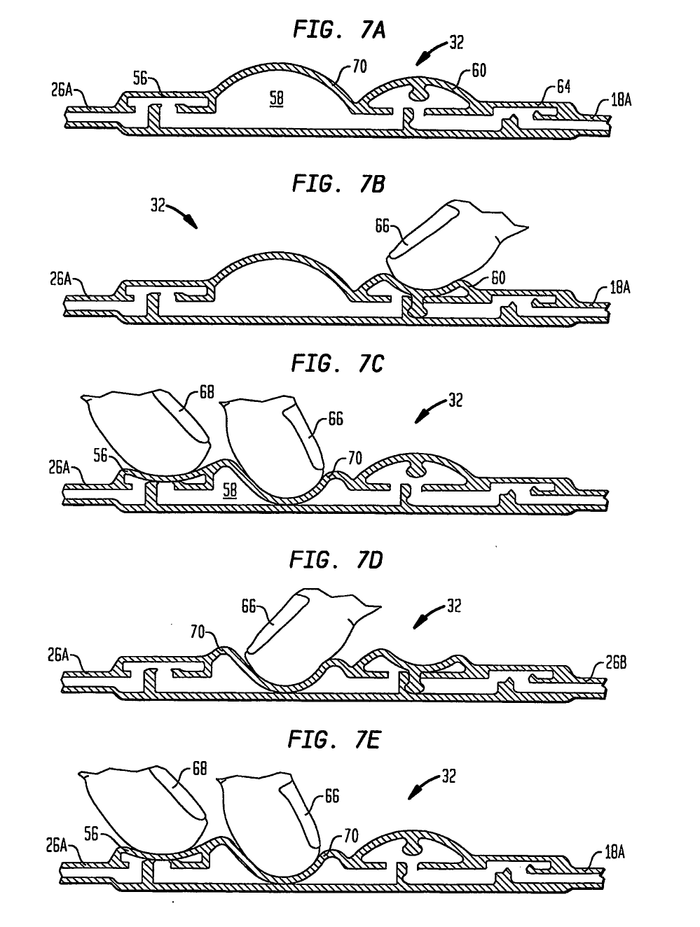

The preferred embodiment of the present invention 30 is shown in FIG. 4. All the components are unitized between the two “Y” connectors, 26A, 26B and 18A and 18B, continuing to the peritoneal catheter 18. Fluid 28 from the cavity 12 in the brain passes through upper tube 26 and to the “Y” connector 26A, 26B and can go either toward an on-off switch 32 with an anti-siphon device 64 or toward the ball-and-cone valve 34 from either pathway, 26A or 26B, to the lower “Y” connector, 18A or 18B, and then through tubing 18 to the peritoneal cavity 36. The “open” or “closed” (or “on” or “off”) setting of the switch 32 will determine the direction of fluid flow. The preferred embodiment of the switch 32 is the catalog number NL850-0155 on /off CSF reservoir with an anti-siphon device such as made by Integra NeuroSciences of Plainsboro, N.J. While that model comprises the preferred on/off device 32, nevertheless, there may be other devices in the prior art that might work just as well. The on-off switch 32 is illustrated in cross-sectional detail in FIG. 6B and is shown as used in FIGS. 7A-7E. FIG. 6B shows the components that comprise the on-off switch 32 which include pathway 26A which passes through occluder 56 which in turn is connected to reservoir 58. Reservoir 58 communicates to the on/off button 60. Downstream from the on/off button 60 is the anti-siphon device 64 which connects to the exiting tubing 18A which in turn is connected to tubing 18 which communicates with the peritoneal cavity 36. Reservoir 58 is covered by a soft depressible skin 70 seen in FIG. 7A. The on/off switch 32 is shown in the “open” or “on” position in FIGS. 6B and 7A. In this mode, the bloody cerebrospinal fluid 28 from ventricle 12 flows through the device 32 unimpeded by obstacles or pressure restraint. Anti-siphon device 64 prevents over drainage of the CSF from the brain ventricle 12. The structure of the anti-siphon device 64, is known in the prior art.

In order to close the one-way switch 32, a physician presses down with his or her finger 66 on the on/off control button 60 as shown in FIG. 7B. The on/off device 32 is located under the scalp 24 of the patient 10 and its features can be accurately determined by the feel of an experienced neurosurgeon. The on/off device 32 remains closed in this position until otherwise opened by the neurosurgeon. It is highly unlikely that patient or staff mishandling could inadvertently cause the switch to open.

FIGS. 7C-7E illustrate the known prior art technique for flushing the various tubes, reservoirs, and the ball-in-cone spring valve as shown in FIG. 4. This will not be described in greater detail except to note that the on/off control button 60 can be popped into its “open” or “on” state by pushing down with the index finger 66 on the top 70 of reservoir 58 while pushing down with a second finger 68 on the occluder 56.

The pressure resistant ball-in-cone spring valve 34 is illustrated in detail in FIG. 6A. Valves such as are known in the prior art and are sometimes referred to as Hakim valves. The preferred embodiment of the invention 30 employs an “OMNISHUNT™ One Piece Valve System Catalog No. 908-322 as manufactured by Integra NeuroScience of Plainsboro, N.J., also the manufacturer of the on/ off switch device 32. The ball-in-cone pressure resistant valve 34 is connected at one end to inlet tubing 26B and at the other end to outlet tubing 18B. The spring pressure against the ball inside of the valve 34 dictates the resistance that it presents to the flow of cerebrospinal fluid 28 through inlet tubing 26B. The preferred pressure resistance is over 40 mm of water and is preferably in the range of 40-80 mm of water.

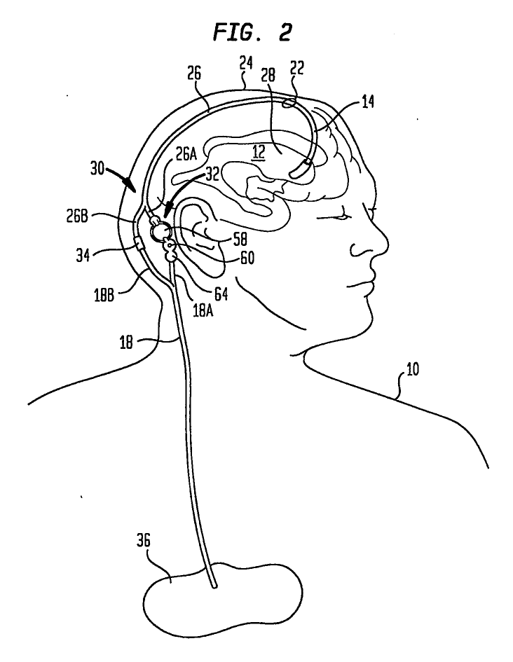

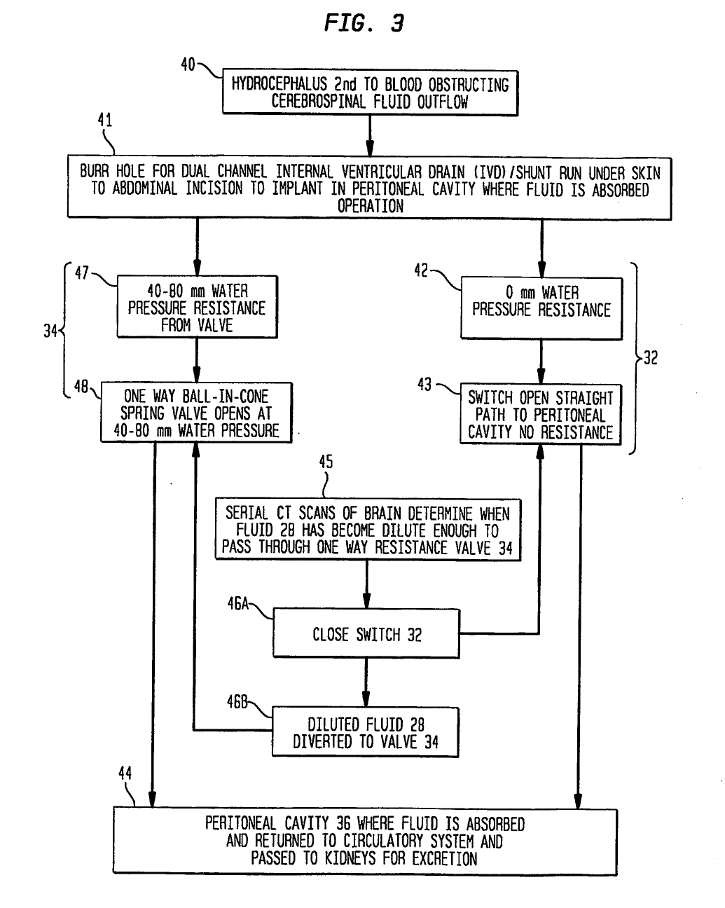

FIG. 2 illustrates the manner in which the invention 30 is inserted into a patient 10 and FIG. 3 graphically illustrates the steps that take place when the invention 30 is employed. Initially a burr hole 22 is drilled into the scalp 24 of patient 10 and a ventricular catheter 14 is inserted through the substance of the brain into the lateral ventricle 12 to drain cerebrospinal fluid 28 in a manner similar to that described with regard to the prior art and illustrated in FIGS. 1A, 1B and 1C. A catheter 26 carries the cerebrospinal fluid 28 under the scalp 24 of the patient 10 and down to an area behind the ear of the patient 10 as illustrated in FIG. 2. The dual-channel shunt device 30 is then attached to catheter 26 at the top end and to catheter 18 of the bottom end which directs cerebrospinal fluid 28 to the peritoneal cavity 36 of the patient 10. Cerebrospinal fluid 28 can be directed either through pathway 26A and the on/off device 32 through pathway 18A to catheter 18, or, it can be directed through 26B and the one-way pressure resistant valve 34 and then through catheter 18B to drainage catheter 18. The method by which this is accomplished is illustrated in further detail in FIG. 3.

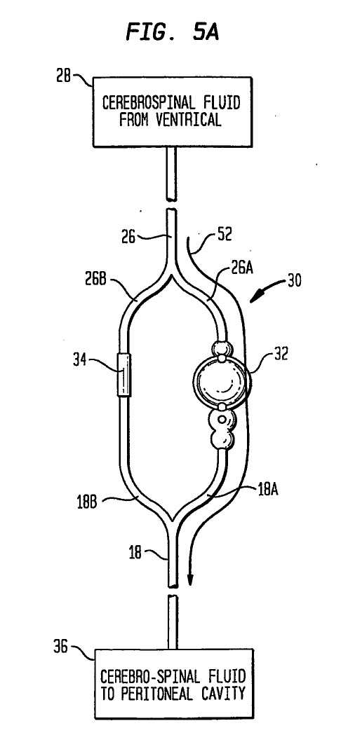

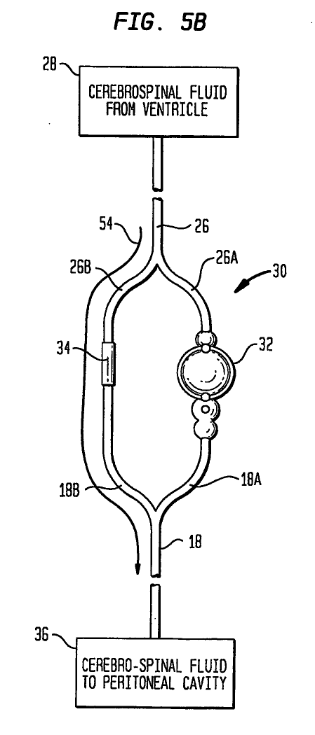

As shown in FIG. 3, if hydrocephalus is detected as shown in step 40, a burr hole 22 is initially drilled through the skull of patient 10 after an incision through the scalp 24 is made as shown by step 41. The plastic ventricular catheter tube is passed through the substance of the brain into the lateral ventricle. Catheter 26 is run under the scalp 24 and attached to the “Y” connector 26A, 26B of the dual-channel shunt device 30, the second “Y” connector 18A, 18B through downstream shunt line 18 to the peritoneal cavity 36 as illustrated by step 41. Because the cerebrospinal fluid 28 is bloody, it is initially directed through the on/off switch 32 with little or no resistance because on/off switch 32 is initially in the “open” state and drains into the peritoneal cavity 36 as shown by steps 42, 43 and 44, respectively in FIG. 3. Under these initial conditions, the cerebrospinal fluid 28 takes the path of least resistance shown by the arrow in FIG. 5A through the on/off switch 32. This comprises the initial setting for the patient 10 with acute hydrocephalus secondary to bloody cerebrospinal fluid. It permits a straight, no-pressure path to the abdominal cavity 36 with no valve resistance involved so that blood blockage can be avoided.

After a period of time the neurosurgeon determines through serial CT scans of the brain whether or not the fluid has become diluted enough so that it can pass through the one-way resistance valve 34, as shown in step 45 of FIG. 3. Once the cerebrospinal fluid 28 is diluted enough, the neurosurgeon closes switch 32 to divert the clearer cerebrospinal fluid 28 through pressure resistance valve 34 as shown in step 46A and 46B of FIG. 3. At that point, the neurosurgeon pushes down with his/her finger 66 through the scalp 24 of the patient 10 onto the on/off control 60 of the switch 32 in the manner illustrated in FIG. 7B. This step plugs the on/off device 32 so that it can no longer conduct the flow of cerebrospinal fluid 28 through pathway 26A and 18A and, instead, diverts the clearer cerebrospinal fluid 28 through the one-way pressure resistance ball-in-cone valve 34. If the pressure of the cerebrospinal fluid 28 on the ball-in-cone pressure resistant valve 34 is above 40 mm of water and preferably in the range of 40-80 mm of water, as shown in step 47, then the one-way ball-in-cone spring valve 34 opens up as shown in step 48 and passes the clearer cerebrospinal fluid 28 to the peritoneal cavity 36 as shown in step 44. The result is that the cerebrospinal fluid 28 then takes the path through the pressure resistant ball-in-cone valve 34 shown by the arrow in FIG. 5B.

In conclusion, the present invention has the following benefits over the prior art such as illustrated in FIGS. 1A and 1B.

First, and of most importance, the device and method proposed eliminates the patient's risk of infection (meningitis and/or ventriculitis) from hospital acquired pathogens. The entire system is implanted within the body with no communication to the outside environment. There is no EVD apparatus for hospital staff to contaminate with bacteria by mishandling.

Second, the implanted device cannot become dislodged by patient movement or staff mishandling, so replacement surgery with its inherent risks is unnecessary.

Third, the Dual Channel IVD/Shunt does not need to be removed after bloody fluid has cleared. No subsequent surgery to insert a ventriculo-peritoneal shunt is necessary for either early or delayed recurrent hydrocephalus. All variables are addressed with the initial surgery.

Fourth, in the common circumstance of re-bleeding after an EVD has been removed, no further surgery is necessary. The Dual Channel IVD/Shunt can be re-opened to straight drainage to the peritoneal cavity by simply pressing the scalp over the on-off switch into the “open” position, thus avoiding another surgery.

Fifth, if the implanted device malfunctions due to obstruction, the Dual Channel IVD/Shunt can be cleared with an injection (saline, heparin etc.) through the scalp into the implanted reservoir rather than with repeat surgery. The device can be palpated to direct the fluid injected into the reservoir toward either the proximal ventricular catheter, the distal peritoneal catheter, or to the ball-in-cone spring valve.

While the invention has been described with reference to the preferred embodiment thereof, it will be appreciated by those of ordinary skill in the art that various modifications can be made to the elements and steps of the invention without departing from the spirit and scope of the invention as a whole.

1. (canceled)

2. The apparatus (30) of claim 11 wherein said pressure resistance valve (34) comprises a ball-in-cone type of spring valve.

3. The apparatus (30) of claim 2 wherein said ball-in-cone type of spring valve (34) presents a pressure gradient in the range of approximately 40-80 millimeters of water resistance to said hydrocephalic fluid.

4. The apparatus (30) of claim 3 wherein said on/off switch (32) comprises:
an occluder section (56) connected to said shunt (14, 26, 26a) from said cavity (12) in the brain;
a reservoir section (58) connected to said occluder section (56);
an on/off control section (60); and,
an anti-siphon section (64) connected between said on/off control section (60) and the section of the shunt (18a) connected to the hydrocephalic fluid receiver (36).

5. The apparatus (30) of claim 4 wherein said hydrocephalic fluid receiver (36) is located within the body of said patient (10).

6. The apparatus (30) of claim 5 wherein said on/off switch (32) is located under the scalp (24) of the patient (10) so that said on/off control section (60) can be turned on and off by pushing down on the scalp (24) of the patient (10).

7. The apparatus (30) of claim 6 wherein a CT scan of the patient's brain is used to determine when the hydrocephalic fluid (28) of the patient is sufficiently clear to turn said on/off switch (32) off thereby diverting substantially clear hydrocephalic fluid (30) through said pressure resistance valve (34).

8. (canceled)

9. The method of claim 12 wherein said step b of determining when said fluid (28) has become sufficiently clear of blood is accomplished by performing a CT scan of the patient's brain.

10. The method of claim 9 wherein said pressure resistant valve (34) offers a pressure approximately in the range of 40-80 Millimeters of water to said fluid (28).

11. An apparatus (30) for draining hydrocephalic fluid (28) from a cavity (12) in the brain of a patient (10) through a shunt line (14, 28, 18) to a fluid receiver (36), said apparatus (30) comprising:
a first pathway having substantially no fluid resistance (26A, 18A) for initially passing said hydrocephalic fluid (28) to said receiver (36) when said fluid (28) includes substantial amounts of blood;
an on/off switch (32) located in said first pathway (26A, 18A);
a second pathway (26B, 18B) connected in parallel with said first pathway (26A, 18A) for passing said hydrocephalic fluid (28) to said fluid reservoir (36) when said fluid (28) is substantially clear of blood; and,
a pressure resistance valve (34) located in said second pathway (26B, 18B) for introducing a substantial amount of resistance to fluid (28) in said second pathway (26A, 16A),
wherein bloody hydrocephalic fluid (28) initially flows through said first pathway (26A, 16A) until it has been determined that said hydrocephalic fluid (28) is substantially clear of blood at which point said on/off switch (32) is turned off and blood free hydrocephalic fluid (28) flow is then diverted from said first pathway (26A, 18A) to said second pathway (26B, 18B) and from there to said fluid receiver (36).

12. A method of shunting hydrocephalic fluid (28) from a cavity (12) in the brain of a patient (10) via a shunt line (14, 26, 18) to a hydrocephalic fluid receiving reservoir (36), said method comprising the steps of:
a. initially passing the flow of said fluid (28), when it contains significant amounts of blood, through an on/off switch (32) located in a first pathway having substantially no fluid resistance (14, 26, 26a, 18a, 18) to said receiving reservoir (36);
b. determining when said fluid (28) has become substantially clear of blood; and,
c. subsequently diverting said clear fluid (28) through a pressure resistant valve (34) located in a second pathway (14, 26, 26b, 18b, 18) parallel to said first pathway (14, 26, 26a, 18a, 18) to said receiving reservoir (36) after it has been determined that said fluid (28) is substantially clear of blood as determined in step b above,
wherein clear fluid (28) passing through said second pathway (14, 26, 26b, 18b, 18) encounters significant resistance up to a predetermined pressure so that said patient experiences substantially normal intracranial pressures after said fluid is substantially clear of blood.
